(19) United States
(12) Patent Application Publication
(10) Pub. No.: US 2006/0263873 A1
(43) Pub. Date: Nov. 23, 2006

(54) Controlled flow microfluidic device and method of fabrication

(76) Inventors: Leanna M. Levine, Redondo Beach, CA (US); Antonio J. Ricco, Los Gatos, CA (US)

Correspondence Address: Robert Nick, POB 3156, Laguna Hills, CA, 92654, US

(21) Appl. No.: 11/436,119

(22) Filed: May 17, 2006

Related U.S. Application Data
(60) Provisional application No. 60/683,103, filed on May 21, 2005.

(51) Int. Cl. C12M 1/34

(52) U.S. Cl. 435/287.2

(57) ABSTRACT

The invention comprises a method and device for cell culture analysis using microporous membranes to control fluid flow in a device. The microporous membranes are of defined porosity and surface area to effectively increase the fluidic circuit resistance and control fluid flow through the device.

### CROSS REFERENCE TO RELATED APPLICATIONS

The present application claims the benefit of priority of Provisional Patent Application Ser. No. 60/683,103 filed May 21, 2005 for Leanna M. Levine et al, the entire content of which is incorporated herein by reference.

### FIELD OF THE INVENTION

The invention comprises a device and method for cell culture analysis using microporous membranes to control fluid flow in the device.

### BACKGROUND OF THE INVENTION

In prior art devices, fluid flow can be difficult to predict and correlate with modeled fluid flow analysis. Flow rates in a device are affected, for example, by changes in the device orientation due to gravitational forces, non-homogeneous surface characteristics of material along the length of the fluidic channels and the general surface material properties of the device related to a specimen contact angle. Accordingly, reproducible microfluidic devices, suitable for use in a commercial, mass production, laboratory setting, are difficult to fabricate.

### BRIEF DESCRIPTION OF THE SEVERAL VIEWS OF THE DRAWINGS

FIG. 1 is an illustrative view of a multiple layer assembly of the invention;

FIG. 2 is an illustrative view of the middle layer of the invention;

FIG. 3 is an illustrative view of a batch fabrication layout of the invention;

FIG. 4 is a cross-section view of a cell culture card of the invention; and

FIG. 5 is a key to FIGS. 1-4 of the invention.

### DETAILED DESCRIPTION OF THE INVENTION

The invention is based on the realization that undesirable changes in fluid flow in a device can be made negligible by introducing into the fluidic network a system of fixed resistances that are greater than the fluid pathway resistances in the device due to the surface properties and geometry of the flow channels.

By placing selected resistances at the inlets, outlets and branches of the flow channels, the same principles that apply to electronic circuit design can be applied to the development of fluidic flow paths where fractional flows in various fluid paths are controlled by the placement of resistive elements. In the embodiments of the invention, porous membranes are employed as resistive elements and such membranes can be used with or without fixed diameter constrictions or vias, to connect one part of a device to another.

The flow in these devices can be modeled using Navier-Stokes flow in which the fluid movement and pressure drops in the system are dependent on the geometry of the fluidic path, the dimensions, and the surface properties of the material, and the properties of the fluid itself (density).

While this model can approximate flow in a system with known geometry, it cannot adequately describe the influence of geometry, fluid composition, and surface for a complex fluidic system, such as the one proposed here. This has resulted in the use of empirical trial and error methods to determine the actual fluid paths in a complex flow pattern.

The addition of porous membranes now requires the fluid flow to be modeled more completely using both Navier-Stokes equations as well as Darcy's law which describes the flow and pressure drops in the system associated with a porous media, such as a membrane, of known porosity and surface properties.

When the pressure drops in the system can be described predominantly using Darcy's law, the flow in the fluidic system can be described and predicted as if the porous structures were resistances in an equivalent electrical circuit. By knowing the resistance to flow, which is the pressure drop/per unit area of membrane at a given flow rate, across the porous structure, the fluid flow in the device can be predicted as the sum or resistances for a set of membranes in series, or if the membranes are in parallel, as the sum of 1/Resistance to flow.

The invention further provides an inexpensive method of placing porous membranes in a microfluidic device to control fluid flow through the device.

In contrast to the fixed diameter orifices used in prior art devices, the porous membranes of the invention provide the primary mechanism of fluid control in a device. Through the distribution of pore sizes, the membranes can create an average property for resistance to flow based on well-known principles in the art of membrane fabrication.

The membranes of the invention also function to provide a large surface area that can be modified either chemically or through adsorption. As such, membrane surfaces can be designed that can swell and change their porosity or surface charge, to provide a means for dynamically adjusting the resistance to flow throughout the device.

In one embodiment, a multiwell fluidic card incorporates microporous membranes at the inlet and outlet of cell-retaining sample wells to control the rate of flow across all the wells. The invention is based on the realization that fluid flow paths and rates in a fluidic device can be passively controlled through the placement of membranes of defined average porosity, surface area and thickness. When the volume and porosities of the membranes are equivalent, the flow rates are the same, regardless of pressure drops in the fluidic channels that lead to each well, as long as there are no other low resistance paths.

Although membranes have been used in microfluidic devices, they have been used to function as a support medium for the exchange or capture of molecular species, or as a valve to permit or reject fluid flow down a specific channel (Frechet, et al). So far, no one has recognized that membranes designed with specific surface areas and porosities solve a general problem in the use of pressure to drive fluids in microfluidic devices.

In prior art devices, small changes in pressure from a pump or vacuum driving fluid through the device and pressure drops experienced in moving liquid down long channels, causes fluid at the outlet of the device to encounter more resistance to flow than fluid at the inlet making it difficult to predict and control fluid movement.

In general, fluid flow in microfluidic devices can be thought of as a circuit, in which liquid will move along the path of least resistance. Control of the fluid flow can be achieved by using active controls, such as valves, that are placed at junctions to resist or allow movement of fluid down a specific channel. These controls can also be used to overcome the inherent pressure drops associated with the fluidic circuit.

As is well known from fluid mechanics, the length and width of channels in a fluidic device can be designed to generate specific pressure drops in order to normalize fluid flow. For microfluidic systems, however, the nature of materials in the walls of the device can contribute significantly to the resistance to flow, making it difficult to predict and control fluid movement.

These effects can be eliminated by introducing into the system a fixed large resistance, such as a porous membrane, to increase the resistance in the fluidic circuit so that fluid flow rates are essentially equivalent in all the wells linked by a single inlet line.

Though membranes have found use as support media as described above, they have not been used as passive controls for fluid movement.

The novel application of membranes of the invention has been demonstrated to simplify the design and implementation of microfluidic fluid circuits for applications in cell culture and in general for the development of multichannel and multiwell devices that are supplied by a single fluid inlet.

The invention permits manufacture of inexpensive microfluidic devices in which a multiwell fluidic card can deliver and remove fluids to each well using a single inlet and single outlet for all the wells. Each well has at the inlet and outlet a porous membrane that contains the cells that are within the well.

The invention also includes a means to integrate membrane placement in a pre-formed substructure, avoiding costly manufacturing steps for the placement of individual membranes at each desired location. This method of fabrication allows much smaller membranes to be produced and permits the density of fluidic channels and features to be expanded to support multiplexed applications.

The invention provides a pre-formed support layer that has defined pores to allow the membrane to form through the depth of the layer and form close contact with the sides to prevent channeling of flow in an uncontrollable fashion.

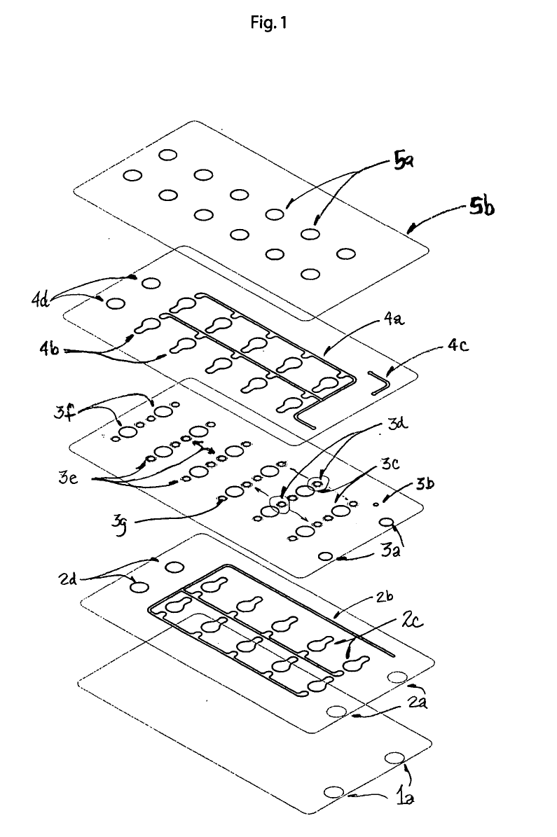

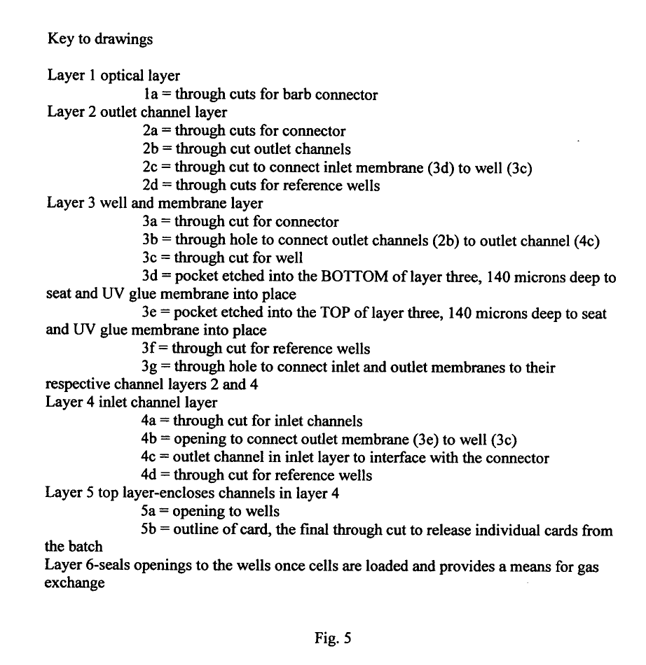

Fabrication of the device can be accomplished by using lamination techniques to form a multilayer structure, as shown in FIGS. 1-5, with a middle layer containing sample wells and through holes to connect fluid flow from the inlet to the outlet. Layers above and below the middle layer include the inlet channels on one side and the outlet channels on the other side. The inlet channels open into a though hole in the middle layer that is separated by the porous membrane. The outlet channels similarly open into a through hole in the middle layer that connects fluid flow through another porous membrane to the outlet. Above and below the channel layers are additional layers that enclose the channels and provide optical windows to the wells.

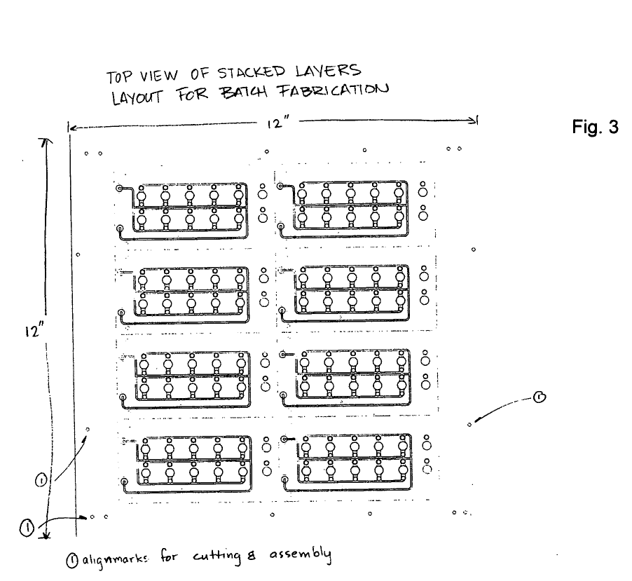

The device is easily fabricated by building out from the middle layer, which permits variation in well depth. The channel layers are symmetrical above and below the middle layer. The layers can be laminated with a pressure sensitive adhesive, a thermal bond adhesive or any of the various bonding methods typically used for joining plastics. A top view of an assembly of stacked layers for batch fabrication is shown in FIG. 3.

The same design of the invention can be fabricated using injection molding techniques instead of forming through lamination. A single injection molded piece with the necessary channels and through holes can be formed with the channels molded directly into the middle layer. The channels can then be enclosed using a number of films laminated to the top and bottom. These films can have optical properties and surface coatings to enhance their function as optical windows, or they can be specialty surfaces for the culture of cells, or contain electrodes and circuits necessary for performing an electrochemical measurement. The layer can also include other sensing elements such as temperature and various gas sensors. The layer can also contain a heating element to control the temperature in the device.

Membranes that can be fabricated at specific locations inexpensively will also allow for the integration of a fluid degassing membrane to eliminate bubble formation in these fluidic devices. Although such membrane degassers are currently available and used for medical applications, specifically to prevent air-bubble formation in blood vessels during infusions, they are made by placing a preformed membrane into a holder and sealing in place using a variety of known methods for joining plastics.

The method of the invention for in-situ fabrication of the membrane will reduce the cost of manufacture and allow for ways of making very small and strategically placed membranes for the purpose of eliminating air bubbles in microfluidic devices.

One embodiment of the invention is a fluidic card used for cell culture in which a single inlet supplies a nutrient solution equally to eight different wells. Outlets from the eight different wells are collected and exit the device from a single outlet port.

The ability to apply controlled resistances in fluidic circuits has general utility in the development of disposable fluidic devices for research, and medical diagnostic applications.

In the absence of a means for scalable in-situ fabrication of membranes, it is currently feasible to cut, pick and place membranes and glue them into the desired location using a variety of methods, including UV or epoxy cure, or other adhesive.

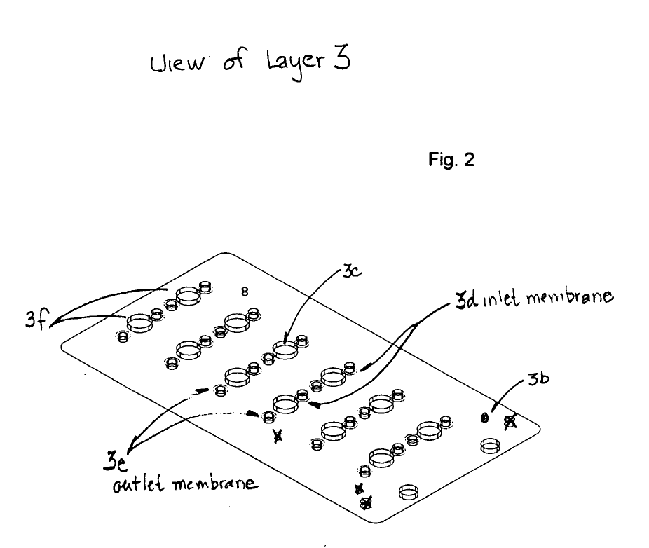

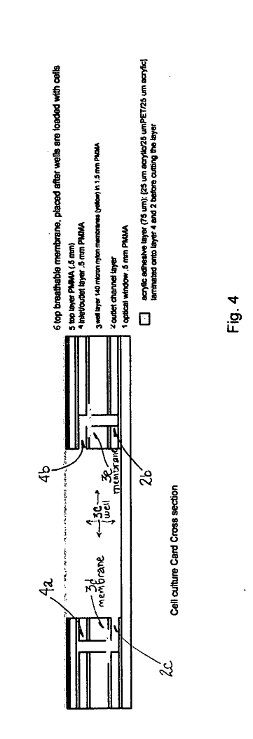

The basic design for the cell culture fluidics card uses the middle layer, as shown in FIG. 2, to form the well and the layer that contains the membrane. The middle layer allows the device to be built from the center out. Each successive layer is laminated on the central layer to form the final device, as shown in FIG. 4.

The membrane is inserted into the middle layer to create a flat, easy to laminate assembly subunit. The inlet membrane is located close to the bottom of the well to allow the device to fill from the bottom and exit from the top to minimize entrapped air bubbles.

The advantage to this design is that for fabrication purposes, and ease of conversion into an injection molded part, the channels are symmetrical around the middle layer.

This basic five layer design can be modified to create many more wells per unit area with membranes creating both a resistance to flow to ensure all the channels and wells fill at the same rate, and to allow the cells to be contained in the individual wells, while conveniently feeding them with a single inlet tube, and having a single outlet to carry away waste products without cross contamination or cross talk between wells.

What is claimed is:

1. A device for cell culture analysis using microporous membranes of defined porosity and surface area to control fluid flow through increased resistance in the fluidic circuit of the device.
